(19) United States
(12) Patent Application Publication
(10) Pub. No.: US 2002/0072056 A1
(43) Pub. Date: Jun. 13, 2002

(54) Method for increasing hybridization efficiency of a nucleic acid

(76) Inventors: Yukio Sudo, Saitama (JP); Hiroshi Shinoki, Saitama (JP); Kousaku Ohkubo, Osaka-fu (JP)

Correspondence Address: Jules E. Goldberg, Esq., Reed Smith LLP, 375 Park Avenue, New York, NY, 10152, US

(21) Appl. No.: 09/804,386

(22) Filed: Mar. 12, 2001

(30) Foreign Application Priority Data
Mar. 10, 2000 (JP) 065862/2000

(51) Int. Cl. C12Q 1/68

(52) U.S. Cl. 435/6

(57) ABSTRACT

According to the present invention, there is provided a method for detecting a sequence of a nucleic acid in a sample which is complementary to a base sequence of a probe nucleic acid which comprises a step of hybridizing the nucleic acid in the sample with the probe nucleic acid, wherein one or more types of bases of the nucleic acid in the sample and/or the probe nucleic acid are chemically modified in advance. According to the method of the present invention, for example, viruses causative of diseases, exogenous genetic substances such as bacteria, or the presence, mutation or expression state of genes carrying particular genetic information of organisms including human, can be detected efficiently.

### FIELD OF THE INVENTION

The present invention relates to a method for increasing efficiency of the detection of a nucleic acid by hybridization. More particularly, the present invention relates to a method for increasing hybridization efficiency in the case where a sequence complementary to a base sequence of a probe nucleic acid is detected in a nucleic acid of a sample by hybridizing the target nucleic acid in the sample with the probe nucleic acid.

### RELATED ART

A method where either one of a target nucleic acid in a sample or a nucleic acid probe comprising a base sequence complementary to the nucleic acid in the target nucleic acid sample is immobilized to a solid phase and hybridized with the other, was developed by E. M. Southern in 1975, and is broadly used as Southern blotting method (a method for detecting a target nucleic acid in an immobilized DNA sample using a nucleic acid probe) or Northern blotting method (a method for detecting a target nucleic acid in an immobilized RNA sample using a nucleic acid probe) (J. Mol. Biol., 98, 503(1975)). In Southern blotting, a nucleic acid sample is subjected to electrophoresis and is immobilized onto a nitrocellulose membrane, nylon membrane or the like, and then is contacted with a nucleic acid probe having a complementary sequence.

Reversely, a method for measuring the amount of a target nucleic acid by immobilizing a nucleic acid probe onto solid phases and contacting it with a target nucleic acid has been developed and are utilized for gene analyses, gene diagnoses and the like.

Based on these techniques, DNA array has been developed as a method for simultaneously measuring amounts of a large number of target nucleic acids existing in a nucleic acid sample, and has been utilized for simultaneous multiple analyses of gene expression and analyses of genetic polymorphism (S. Fodor, Science 277, 393(1997); Nature Genetics Supplement 21:20 (1999)).

In either of the above methods, formation of hybrids by hybridization of nucleic acids complementary to each other is utilized. In these hybrids, the higher complementarity of base sequences is, more strongly target nucleic acid fragments in the samples bind to probe nucleic acids, thereby the bonds become not to dissociate even at a higher temperature. Thus, whether the target nucleic acid in the sample has a complete complementarity to the probe nucleic acid can be determined by washing the hybrid under the condition where the hybrid is not dissociated when the target nucleic acid and the probe nucleic acid have a complete complementarity with each other and it is dissociated when its complementarity is incomplete. The condition is controlled by a temperature, an ionic strength or the like.

It is necessary that the conditions of reaction and dissociation are optimized since binding energy of hybrids is varied depending on their base sequences even in the case of completely complementary nucleic acids. In oligo DNA of approximately 20 bases, their binding energy can be estimated by base contents of the DNA (GC contents) (Molecular Cloning: A Laboratory Manual, 2nd Ed., Section 9, Cold Spring Harbor Laboratory, Cold Spring Harbor, N.Y., 1989). However, when large DNA such as cDNA are subjected, it is believed that hybrid formation is greatly affected by formation of a tertiary structure by hydrogen bond in the DNA molecules. Since it is difficult to presume formation of intramolecular tertiary structure from base contents, optimization of hybridization conditions becomes difficult. Also, there is a problem that the formation of intramolecular tertiary structure by hydrogen bonds adversely affects hybridization efficiency and reduces detection sensitivity.

### SUMMARY OF THE INVENTION

The present invention aims at dissolving the problems of the prior art described above. The present invention aims at providing a method for increasing hybridization efficiency by preventing the formation of tertiary structure by hydrogen bonds in the nucleic acid molecules.

As a result of studying extensively in order to dissolving the problems described above, the present inventors have found that the efficiency of hybridization can be increased by chemically modifying one or more types of bases of a nucleic acid in a sample and/or a probe nucleic acid prior to hybridization of the nucleic acid with the probe nucleic acid, and have completed the present invention.

According to the present invention, there is provided a method for detecting a sequence of a nucleic acid in a sample which is complementary to a base sequence of a probe nucleic acid which comprises a step of hybridizing the nucleic acid in the sample with the probe nucleic acid, wherein one or more types of bases of the nucleic acid in the sample and/or the probe nucleic acid are chemically modified in advance.

Preferably in the present invention, one or more types of bases of the nucleic acid in the sample and/or the probe nucleic acid are chemically modified in advance, thereby reducing the intensity of an intramolecular hydrogen bond of the nucleic acid.

Preferably in the present invention, the nucleic acid in the sample is DNA.

Preferably in the present invention, the probe nucleic acid is probe DNA.

Preferably in the present invention, the chemically modified base is cytidine or guanine.

In one embodiment of the present invention, the bases are chemically modified by treating the nucleic acid with hydrazine.

Preferably in the present invention, the probe nucleic acid is immobilized on a support.

Preferably in the present invention, various probe nucleic acids are immobilized on a support.

According to another aspect of the present invention, there is provided a method for reducing the intensity of an intramolecular hydrogen bond of a nucleic acid, which comprises a step of treating the nucleic acid with hydrazine or dimethyl sulfate.

According to still another aspect of the present invention, there is provided a reagent kit for increasing hybridization efficiency which is used to perform the method according to claim 1, which comprises hydrazine and/or dimethyl sulfate as a reagent for chemically modifying the bases in the nucleic acid.

### DETAILED DESCRIPTION OF THE INVENTION

The embodiments and examples of the present invention are described below.

In the method of the present invention, the sequence complementary to the base sequence of the probe nucleic acid is detected in the nucleic acid in the sample by hybridizing the nucleic acid in the sample with the probe nucleic acid.

The term “nucleic acid” used herein is used to mean that it includes both DNA and RNA, and nucleic acids may be naturally occurring nucleic acids, nucleic acids produced by gene recombinant techniques, and chemically synthesized nucleic acids.

The term “nucleic acid in sample” used herein is the nucleic acid which comprises a target sequence with which a probe nucleic acid can be hybridized. The type of the sample can include, but are not limited to, any samples such as samples derived from organisms, samples from cultured cells, or samples from artificially constructed DNA library. When the nucleic acid in the sample has an extremely large molecular weight such as those derived from chromosomes, they may be cleaved into an appropriate size in length by an appropriate restriction enzyme.

The “probe nucleic acid” used herein mean the nucleic acid which has an unique base sequence to be detected (for example, a particular gene or its partial base sequence). The type of the probe nucleic acids includes, but are not limited to especially, for example, cDNA clones and oligonucleotides, and their lengths are not limited especially. Those having any length from several base pairs to dozens kilobase pairs or more can be used.

In the method of the present invention, a sequence in the nucleic acid in the sample which is complementary to the base sequence of the probe nucleic acid is detected. Detection includes detection of the presence or absence of a complementary sequence, as well as detection of an existing amount of the complementary sequence and detection of mutations in the complementary sequence.

It is desirable that either one of the probe nucleic acid or the nucleic acid in the sample is labeled (generally, a nucleic acid other than an immobilized nucleic acid) in order to detect if a probe nucleic acid is hybridized with a target sequence of a nucleic acid in the sample after hybridization. Types and procedures of labeling are known to those skilled in the art, and either of radiolabeling or non-radiolabeling can be chosen appropriately. The examples of radiolabeling include labeling of nucleic acids with radioisotopes such as ³H, ¹²⁵I, ³⁵S, ¹⁴C or ³²P. The examples of non-radiolabeling include fluorescence labeling (digoxigenin, etc.) or enzyme labeling (alkali phosphatase labeling via biotin-avidin system, etc.).

The method of the present invention comprises hybridization of a nucleic acid in a sample with a probe nucleic acid. The way of hybridization are not limited, and can be performed in any way including Southern hybridization, Northern hybridization, dot hybridization, colony hybridization, plaque hybridization and the like. Although in the processes mentioned above, one of the nucleic acids is immobilized and then hybridized with the other of the nucleic acids. In the method of present invention, both of the nucleic acids can be hybridized in solution without immobilizing the nucleic acid.

The conditions for hybridization can be appropriately set depending on a length of the base sequence to be detected and a base content (GC content) thereof. For example, hybridization can be carried out in a buffer of an appropriate ionic strength at 42. C to 68. C using a filter on which a sample nucleic acid is immobilized. After completion of hybridization, the filter is washed with buffer of an appropriate ionic strength at 42. C to 68. C. The target nucleic acid in the sample can be detected by detecting the probe nucleic acid which was hybridized with the target nucleic acid in the sample using the filter after washing. The above mentioned conditions for hybridization and washing are only examples. General manipulations and conditions for hybridization are described in, for example, Molecular Cloning: A Laboratory Manual, 2nd Ed., Section 9 (Cold Spring Harbor Laboratory, Cold Spring Harbor, N.Y., 1989).

Optimal selection of the reaction temperature and the ionic strength is required for precise formation of hybrids between complementary sequences in hybridization. If the temperature is too high, the probe can not bind to the nucleic acid having a complementary sequence. If the temperature is too low, the probe binds to the nucleic acid nonspecifically. In order to detect the base sequence complementary to the probe more precisely, it is important that unstable hydrogen bonds are eliminated to wash out nonspecifically bound probes and mismatched probes, by lowering the ionic strength (salt concentration) in the solution or by increasing the temperature of the solution. Try and error are generally needed to set a proper condition for hybridization and washing.

Especially, a precision which can eliminate mismatch at one base pair level is required for the hybridization and washing condition used in gene diagnosis.

The method of the present invention is characterized in that one or more types of bases in the nucleic acid in the sample and/or the probe nucleic acid are chemically modified prior to hybridization.

The nucleic acid of which bases are chemically modified may be the nucleic acid in the sample or the probe nucleic acid, and either one or both may be modified.

The chemically modified base may be any of adenine (A), cytidine (C), guanine (G), thymine (T), or uridine (U). Cytidine (C) or guanine (G) is preferable in the light of preventing the formation of intramolecular hydrogen bonds efficiently, and cytidine (C) is especially preferable. When two types of nucleic acids which are complementary with each other are hybridized, binding between GC is stronger than that between AT because an AT pair is formed by two hydrogen bonds whereas a GC pair is formed by three hydrogen bonds. Therefore, prevention of GC binding pairs by chemical modification of G or C can prevent intramolecular hydrogen bonds more efficiently.

The method for chemically modifying bases is described below.

Nucleic acids such as DNA and RNA form hybrids with nucleic acids having a complementary sequence. These hybrids are believed to be formed by hydrogen binding forces at base moieties (purine or pyrimidine) of a nucleic acid in DNA or RNA. Thus, it is possible to break portions of complementary bindings if purine or pyrimidine base can be specifically modified. An example of the methods for specific modification of nucleic acid bases includes Maxam-Gilbert method which is utilized for a DNA sequencing (Molecular Cloning, 2nd Ed., Vol. 2, Section 13). For example, hydrazine can modify pyrimidine bases specifically, however, only cytosine is modified to open the ring of pyrimidine bases in the presence of a high concentration of salts. Also, dimethyl sulfate can modify purine bases of guanine. Cytosine or guanine can be specifically modified using these reagents.

An alternative method for modifying bases includes a method using an intercalator specific for a base. Concretely, the method using an intercalator derivative such as pyrene can be used.

In the method of the present invention, the intensity of hydrogen bonds in the nucleic acid molecule is reduced by chemically modifying bases of the nucleic acid prior to hybridization as described above. By reducing the intensity of hydrogen bonds in the nucleic acid molecule in this way, a formation of a tertiary structure via intramolecular folding of the nucleic acid can be prevented, and hybridization efficiency can be increased.

In a preferred embodiment of the present invention, either one of the nucleic acid in the sample or the probe nucleic acid is immobilized onto a support (solid phase support, e.g., filter), and the other nucleic acid is hybridized. In a more preferred embodiment, the probe nucleic acid is immobilized onto the support. It becomes possible that various unique base sequences are simultaneously detected and quantified in the sample nucleic acid by using a DNA array where various nucleic acids are immobilized as a probe nucleic acid onto the support and hybridizing the array with the nucleic acid in the sample.

By using the hybridization method of the present invention, any desired base sequence of nucleic acids (DNA or RNA) in viruses, microbes, animals, plants and human, can be detected, identified and quantified, and the presence or absence of mutations in various base sequences can be detected.

According to another aspect, the present invention relates to a method for reducing the intensity of an intramolecular hydrogen bond of a nucleic acid, which comprises a step of treating the nucleic acid with hydrazine or dimethyl sulfate. As mentioned above, hydrazine can modify pyrimidine bases specifically, however, only cytosine is modified to open ring of the pyrimidine bases in the presence of a high concentration of salts. Also, dimethyl sulfate can modify purine base of guanine. Cytosine or guanine in nucleic acids can be specifically modified using these reagents, thereby reducing the intensity of intramolecular hydrogen bonds of nucleic acids and increasing hybridization efficiency.

According to still another aspect, the present invention relates to a reagent kit for increasing hybridization efficiency which comprises hydrazine and/or dimethyl sulfate as a reagent for chemically modifying the bases. As mentioned above, since cytosine or guanine can be chemically modified specifically by treating nucleic acids using hydrazine and/or dimethyl sulfate, these are useful as a reagent for increasing hybridization efficiency. These reagents for chemical modification of bases can be appropriately combined with other reagents required for nucleic acid hybridization to prepare a kit for high efficiency hybridization. The examples of other reagents include reagents required for labeling nucleic acids (e.g., labeling materials, and reagents needed for nucleic acid labeling reactions).

The contents of Japanese Patent Application No. 2000-65862 on which the present application claims a priority based, is incorporated herein by reference.

The present invention is described more concretely by the following examples. The materials, reagents, ratios, manipulations and the like shown in the following examples can be appropriately altered without departing from the spirit of the invention. Therefore, the scope of the invention is not limited to the illustrative examples shown below.

### EXAMPLES

### Example 1

### Preparation of Labeled CDNA

³²S-dATP-labeled cDNA was prepared by Ready-to-Go DNA labeling beads (Amarsham) using human CDNA library (human lung cDNA; Takara Co.,) as a template (This cDNA is referred to as labeled cDNA I.). 15. l of 5 M NaCl and 30. l of 95% hydrazine solution were added to 10. l of this labeled cDNA (5. g of DNA content), and the mixture was left at 20. C for 5 minutes to perform modification of bases.

200. l of 0.3 M sodium acetate solution was added to the reaction mixture, and the mixture was stirred at 4. C to stop the reaction, and then 750. l of ethanol was added. The reaction mixture was subjected to ethanol precipitation at −20. C to purify DNA (This DNA is referred to as labeled CDNA II).

### Example 2

### Preparetion of GAPDH cDNA Immobilized Membrane

Human GAPDH cDNA was amplified by a PCR method using CDNA prepared in Example 1 as a template. The primers of the following sequences were used.

forward primer: ATG GGG AAG GTG AAG GTC GGA G
reverse primer: TTA CTC CTT GGA GGC CAT GTG G

The PCR product was subjected to electrophoresis on 1% low melting agarose gel. The corresponding band (1007 bp) was recovered and was amplified again by PCR. The product was purified again by electrophoresis on 1% low melting agarose gel, and the concentration of the purified product was measured by a spectrometer. This cDNA solution was diluted at 10 ng/. l with TE buffer, and then 2. l of the aliquot was blotted on a nylon membrane (HybondN+, Arnarsham). The membrane was dried and irradiated with UV light to prepare a GAPDH cDNA immobilized membrane.

### Example 3

The GAPDH cDNA immobilized membrane prepared in Example 2 was immersed in 30 ml of the hybridization buffer (5×SSC buffer containing 0.1% SDS and 5% dextransulfate) at 60. C for 30 minutes. Then, the labeled cDNA I or labeled cDNA II prepared in Example 1, which had been heated at 95. C for 5 minutes followed by rapid cooling with ice, was added and the hybridization was performed at 65. C for 18 hours.

After completion of hybridization, the membrane was washed with SSC containing 0.1% of SDS twice and with 0.5×SSC containing 0.1% of SDS twice, and was wrapped with plastic wrap, and was exposed to the imaging plate (Fuji Photo Film Co. Ltd.) for 10 hours. Then, the radioactivity was measured by using BAS 1800.

The measured values obtained by subtracting background (RI relative intensity) were 800 for the labeled cDNA I and 3,300 for the labeled cDNA II, showing that hybridization efficiency was increased more effectively by using hydrazine-treated cDNA II.

According to the method of the present invention, hybridization efficiency can be increased in the case where a sequence complementary to a base sequence of a probe nucleic acid is detected in a nucleic acid in a sample by hybridizing the target nucleic acid in the sample with the probe nucleic acid. According to the method of the present invention, for example, viruses causative of diseases, exogenous genetic substances such as bacteria, or the presence, mutation or expression state of genes carrying particular genetic information of organisms including human, can be detected efficiently.

What is claimed is:

1. A method for detecting a sequence of a nucleic acid in a sample which is complementary to a base sequence of a probe nucleic acid which comprises a step of hybridizing the nucleic acid in the sample with the probe nucleic acid, wherein one or more types of bases of the nucleic acid in the sample and/or the probe nucleic acid are chemically modified in advance.

2. The method according to claim 1 wherein one or more types of bases of the nucleic acid in the sample and/or the probe nucleic acid are chemically modified in advance, thereby reducing the intensity of an intramolecular hydrogen bond of the nucleic acid.

3. The method according to claim 1 wherein the nucleic acid in the sample is DNA.

4. The method according to claim 1 wherein the probe nucleic acid is probe DNA.

5. The method according to claim 1 wherein the chemically modified base is cytidine or guanine.

6. The method according to claim 1 wherein the bases are chemically modified by treating the nucleic acid with hydrazine.

7. The method according to claim 1 wherein the probe nucleic acid is immobilized on a support.

8. The method according to claim 1 wherein various probe nucleic acids are immobilized on a support.

9. A method for reducing the intensity of an intramolecular hydrogen bond of a nucleic acid, which comprises a step of treating the nucleic acid with hydrazine or dimethyl sulfate.

10. A reagent kit for increasing hybridization efficiency which is used to perform the method according to claim 1, which comprises hydrazine and/or dimethyl sulfate as a reagent for chemically modifying the bases in the nucleic acid.


SEQUENCE LISTING (2 sequences)

SEQ ID NO: 1 - DNA, 22 bp, Artificial Sequence
  Artificially Synthesized Primer Sequence (Forward Primer)
atggggaaggtgaaggtcggag

SEQ ID NO: 2 - DNA, 22 bp, Artificial Sequence
  Artificially Synthesized Primer Sequence (Reverse Primer)
ttactccttggaggccatgtgg